(19) United States
(12) Patent Application Publication
(10) Pub. No.: US 2026/0283618 A1
(43) Pub. Date: Sep. 24, 2026

(54) ENDOSCOPIC PURSE STRING SUTURE SURGICAL DEVICE

(71) Applicant: INTUITIVE SURGICAL OPERATIONS, INC., Sunnyvale, CA (US)

(72) Inventors: Paul Millman, San Jose, CA (US); William Burbank, Sandy Hook, CT (US); Nina Frankel, Capitola, CA (US); Steven D. Wexner, Parkland, FL (US)

(73) Assignee: INTUITIVE SURGICAL OPERATIONS, INC., Sunnyvale, CA (US)

(21) Appl. No.: 19/685,788

(22) Filed: May 22, 2026

Related U.S. Application Data
(63) Continuation of application No. 18/737,262, filed on Jun. 24, 2024, now Pat. No. 12,642,528.
Application No. 18/737,262 is a continuation of application No. 17/281,578, filed on Oct. 18, 2019, now Pat. No. 12,029,426.
(60) Provisional application No. 62/747,912, filed on Oct. 19, 2018.

(51) Int. Cl. A61B 17/115; A61B 17/00; A61B 17/11; A61B 34/37

(52) U.S. Cl. CPC A61B 17/115; A61B 2017/00367; A61B 2017/1142; A61B 34/37

(57) ABSTRACT

A surgical stapler for applying a suture to tissue includes a first jaw and a second jaw configured to receive a cartridge. An actuation mechanism translates a drive member distally through the first and second jaws to apply staples to tissue such that a suture, in combination with the staples, forms a purse string when the stapler activated. The cartridge includes a first upper portion and a second lower portion.

### CROSS-REFERENCE TO RELATED APPLICATIONS

This application is a continuation of U.S. Nonprovisional Application No. 18/737,262 filed Jun. 24, 2024, which is a continuation of U.S. Nonprovisional Application No. 17/281,578 filed Mar. 30, 2021, Issued as U.S. Patent No. 12,029,426 on Jul. 9, 2024, which is a National Stage of International Application No. PCT/US2019/056979 filed Oct. 18, 2019, which claims benefit of U.S. Provisional Application No. 62/747,912, filed Oct. 19, 2018, the entire disclosure of which is incorporated herein by reference for all purposes.

### BACKGROUND

Surgical anastomosis joins together two hollow organs, usually to restore continuity after resection, or less commonly to bypass an unresectable disease process. Anastomosis is typically performed on blood vessels including arteries and veins; gastrointestinal tract including esophagus, stomach, small intestine, colon, rectum, anus, bile ducts and pancreas; or urinary tract including ureters, urinary bladder and urethra, and fallopian tubes.

Purse string sutures and purse string appliers may be used during anastomosis procedures. A suture is typically placed using a needle, staples or other suitable means for attaching the suture to the tissue. After attachment, the ends of the suture remain loose for pulling to contract or close the tissue.

In minimally invasive, e.g. endoscopic, surgical anastomosis procedures, it would be advantageous to provide an endoscopic purse string device which could apply purse string sutures in a minimally invasive manner and that is compatible with a robotic surgical system.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure relates to surgical staplers for applying a suture to tissue having a cartridge including a shuttle configured to engage a series of staple drivers for ejecting staples upon distal translation of a drive member.

In another aspect, the present disclosure relates to surgical staplers for applying a suture to tissue including a cartridge having a first upper portion and a second lower portion connected by a connecting member and configured to be installed along the longitudinal axis of the end effector.

In another aspect, the present disclosure relates to a jaw-closing mechanism having a first and second link member secured to a first and second jaw, the first and second links coupled with a first and second pair of drive cables configured to apply force to the first and second link members to cause them to rotate about pivot pins and ride through a cam slot having a proximal portion for parallel closure of the jaws and a distal portion for angular closure of the jaws.

In embodiments, a surgical stapler for applying a suture to tissue in accordance with this disclosure includes an elongated shaft having a distal end and a proximal end. The end effector defines a longitudinal axis and includes a first jaw and a second jaw. The first jaw and second jaw are configured to receive a cartridge and to move from an open position to a closed position to apply staples to tissue such that a suture, in combination with the staples, form a purse string with the tissue when the stapler is activated. The surgical stapler further includes a drive member configured to translate distally and retract proximally through the end effector, and an actuation mechanism configured to translate the drive member distally through the end effector and retract the drive member proximally through the end effector. The cartridge includes a first upper portion configured to fit into the first jaw and a second lower portion configured to fit into the second jaw. The cartridge further includes a shuttle configured to engage a series of staple drivers for ejecting staples upon distal translation of the drive member In embodiments, the first upper portion and second lower portion are connected by a connecting member, and the cartridge is configured to be installed along the longitudinal axis of the end effector.

In another aspect, a jaw-closing mechanism for a surgical stapler for applying suture to tissue in accordance with this disclosure relates to a first link member and a second link member. The first and second link members are configured to rotate about a pair of pivot pins upon application of force to the first and second link members. A pair of outer pins are configured to secure the first and second link members to a first jaw and a second jaw. A first and second pair of drive cables are configured to apply force to the first and second links to cause rotation about the pivot pins. The jaw-closing mechanism further includes a cam slot formed on one of the jaws configured to accept a cam slot pin of at least one of the link members. The cam slot has a proximal portion for parallel closure of the jaws, and a distal portion for angular closure of the jaws.

### BRIEF DESCRIPTION OF THE DRAWINGS

The above and other aspects, features, and advantages of the present surgical instruments will become more apparent in light of the following detailed description when taken in conjunction with the accompanying drawings in which:

FIG. 1 is a perspective view of an illustrative surgical instrument in accordance with the present disclosure;

FIG. 1A is a perspective view of the distal end portion of the surgical instrument of FIG. 1 shown having an end effector mounted to an elongated shaft and a backend mechanism configured to actuate the instrument;

FIG. 2 is a perspective exploded view of a staple cartridge suitable for use with the surgical instrument of FIG. 1;

FIG. 2A is a perspective view showing installation of a cartridge into the surgical instrument of FIG. 1;

FIGS. 2B illustrates a portion of an illustrative surgical instrument in accordance with this disclosure having a flexing latch;

FIG. 2C illustrates a portion of an illustrative cartridge suitable for use with the surgical instrument of FIG. 1;

FIG. 2D illustrates an illustrative jaw suitable for use with the surgical instrument of FIG. 1 having hooks configured to engage and retain the cartridge of FIG. 2C within the jaws;

FIG. 2E illustrates an exploded view of another illustrative jaw and cartridge suitable for use with the surgical instrument of FIG. 1;

FIG. 3 is a partial perspective view of a portion of the end effector with a fresh reload installed;

FIG. 4 is a perspective view of the distal end portion of the surgical instrument of FIG. 1 with the jaws in the angled open position;

FIG. 5 is a perspective view of the distal end portion of the surgical instrument of FIG. 1 with the jaws in the parallel position;

FIG. 6 is a perspective view of the distal end portion of the surgical instrument of FIG. 1 with the jaws in the completely closed position;

FIG. 7 is a cross-sectional perspective view of the jaw closure mechanism of the surgical instrument of FIG. 1;

FIG. 8 is a perspective view of the first and second links of the jaw closure mechanism;

FIG. 9 is a perspective view of a portion of a jaw of the surgical instrument of FIG. 1;

FIG. 10 is a perspective view of the articulation mechanism of the surgical instrument of FIG. 1 with parts removed to reveal internal structures;

FIG. 11 is a perspective view of the shuttle of the surgical instrument of FIG. 1;

FIG. 11A is a partial perspective view with parts removed showing an unfired cartridge installed in the surgical instrument of FIG. 1;

FIG. 12 is a cross-sectional perspective view of the drive mechanism of the surgical instrument of FIG. 1 with parts removed showing a drive member configured to drive distally upon actuation;

FIG. 13 is a partial perspective view with parts removed of a partially driven shuttle contacting a staple driver as it moves distally upon actuation;

FIGS. 13A and 13 B schematically show the firing of a staple to capture the suture and form a purse string;

FIG. 14 depicts an illustrative vessel that has been severed and secured with a series of staples combined with sutures to form purse strings;

FIG. 15 depicts a locking mechanism suitable for use in the surgical instrument of FIG. 1 in a disabled configuration;

FIG. 16 depicts the locking mechanism of FIG. 15 in an enabled configuration;

FIG. 17 illustrates a top view of an operating room employing a robotic surgical system utilizing aspects of the present disclosure; and

FIG. 18 illustrates a simplified side view of a robotic arm assembly that is usable with various aspects of the present disclosure.

### DETAILED DESCRIPTION

Particular embodiments of the present surgical instruments are described hereinbelow with reference to the accompanying drawings; however, it is to be understood that the disclosed embodiments are merely exemplary of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in any unnecessary detail.

While the following disclosure is presented with respect to a surgical instrument for forming purse strings, it should be understood that certain features of the presently described surgical instruments may be readily adapted for use in any type of surgical clamping, cutting, or sealing instruments. The surgical clamping and cutting instrument may be a minimally invasive (e.g., laparoscopic) instrument or an instrument used for open surgery.

Additionally, the features of the presently described surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

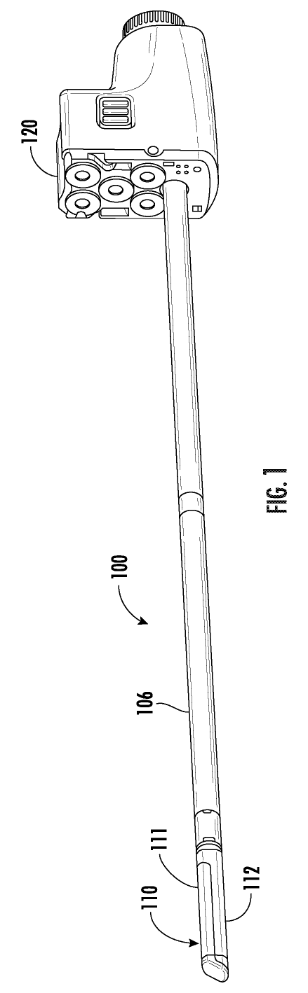

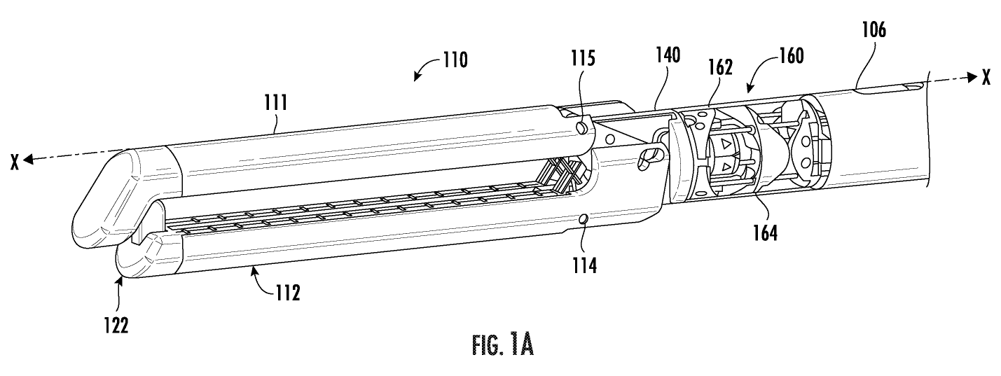

FIG. 1 is a perspective view of an illustrative surgical instrument 100 in accordance with embodiments of the present disclosure having a backend mechanism 120, and an end effector 110 mounted on an elongated shaft 106. Backend mechanism 120 typically provides a mechanical coupling between the drive tendons or cables of the instrument and motorized axes of the mechanical interface of a drive system. Further details of known backend mechanisms and surgical systems are described, for example, in U.S. Pat. No. 8,597,280, U.S. Pat. No. 7,048,745, and U.S. Pat No. 10,016,244. Each of these patents is hereby incorporated by reference in its entirety.

Actuation mechanisms of surgical instrument 100 employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. No. 7,666,191 and U.S. Pat No. 9,050,119 both of which are hereby incorporated by reference in their entireties.

FIG. 1A shows the distal end portion of surgical instrument 100, including an end effector 110 defining a longitudinal axis X-X and having a first jaw 111, a second jaw 112, a cartridge 122, a clevis 140 for mounting jaws 111, 112 to the instrument, and an articulation mechanism, such as wrist 160. First and second jaws 111, 112 are configured to move from an open position to a closed position. In the closed position, jaws 111, 112 cooperate to clamp tissue.

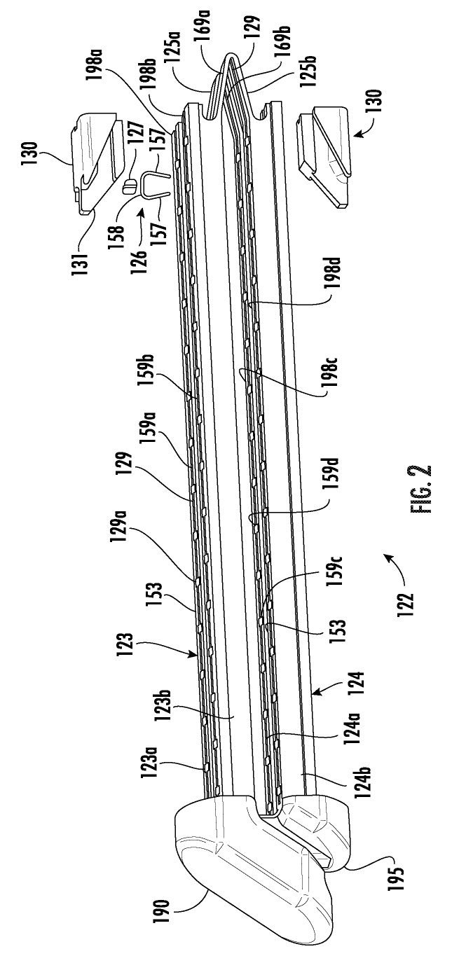

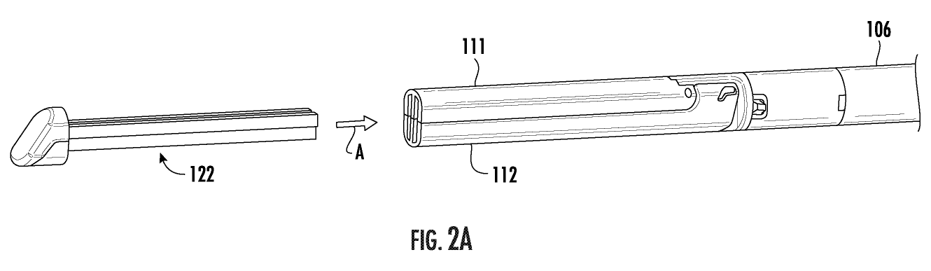

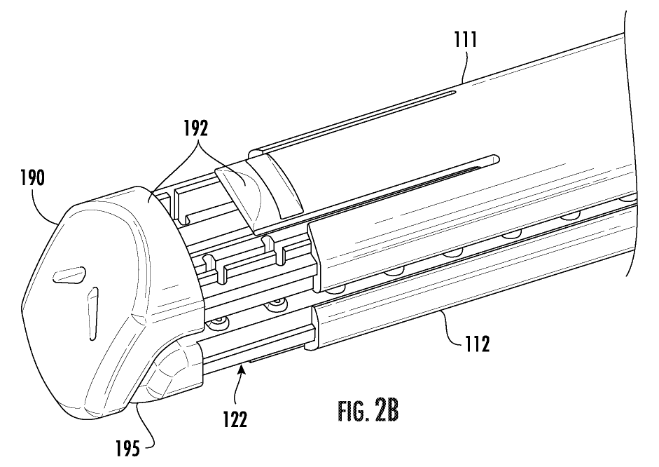

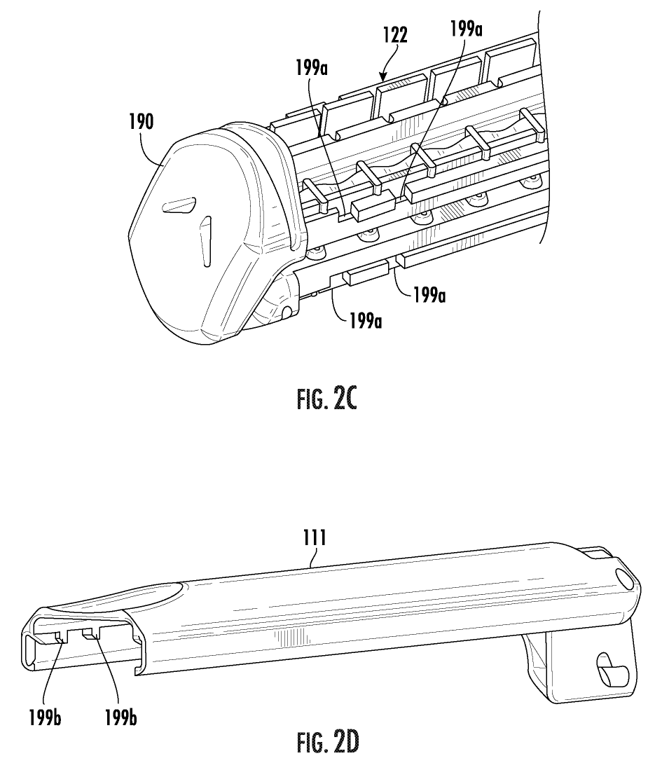

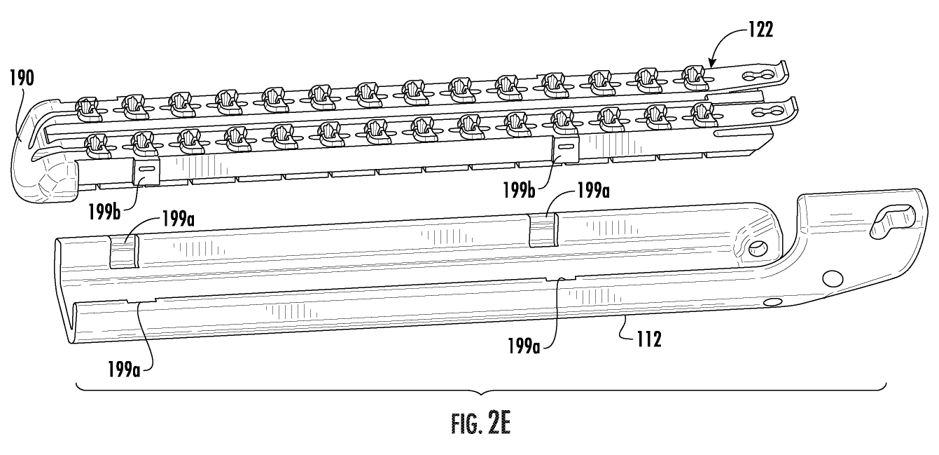

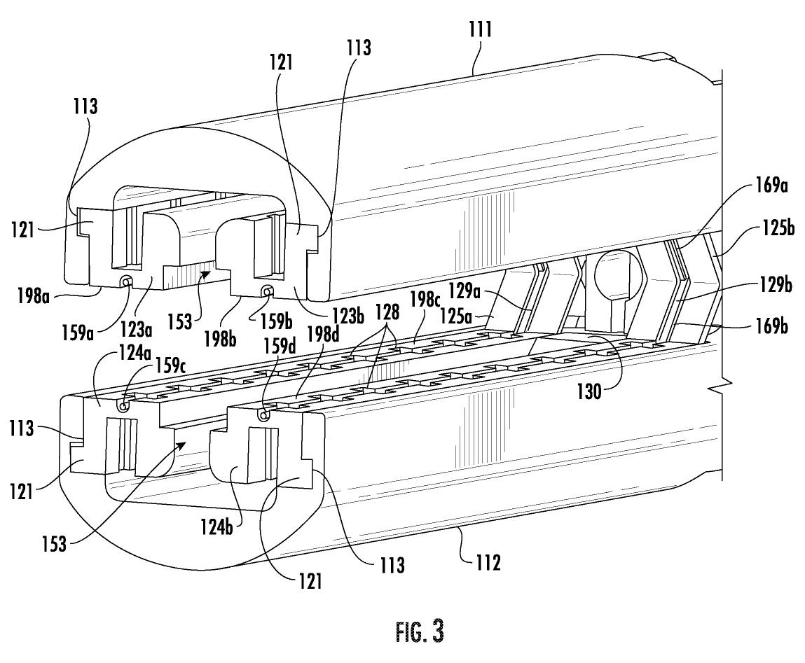

FIG. 2 depicts cartridge 122. In embodiments, cartridge 122 includes an upper portion 123 having a first and second side 123a, 123b, and a lower portion 124 having a first and second side 124a, 124b. Each pair of first and second sides 123a, 123band 124a, 124b is separated by a center knife channel 153. Each side 123a, 123b and 124a, 124bdefines a tissue contacting surface 198a, 198b and 198c, 198d having staple retaining pockets 128 and a suture retaining channel 159a-d formed therein. (See also FIG. 3.) In embodiments, tissue contacting surfaces 198a-d may further include protrusions (not shown) positioned about staple receiving pockets 128. Protrusions help to further secure clamped tissue and to resist movement that might be induced from forces created by the severing and stapling of clamped tissue. Staples 126 are supported on corresponding staple drivers 127 provided within respective staple receiving pockets 128 formed in cartridge 122. Staple receiving pockets 128 may be cutouts that are substantially perpendicular to the longitudinal axis of end effector 110, and similar in length to the desired size of staples 126 to be fired. Each staple 126 include legs 157 and a backspan 158. Suture retaining channels 159a-d run substantially parallel to the longitudinal axis of end effector 110 and are substantially aligned with the center of staple receiving pockets 128 so that upon firing of staples 126, sutures 129a, 129b are captured at various locations between staples 126 and tissue 197 as described in more detail below.

Upper portion 123 and lower portion 124 of cartridge 122 are configured to be contained within first jaw 111 and second jaw 112, respectively. Side 123a of upper portion 123 is connected to side 124a of lower portion 124 at the proximal end of cartridge 122 by living hinge 125a. Likewise, side 123bof upper portion 123 is connected to side 124b of lower portion 124 at the proximal end of cartridge 122 by living hinge 125b. Channels 159a, 159c of sides 123aand 124a of cartridge 122 extend to living hinge 125a and align with living hinge channel 169a. Likewise, channels 159b,159d of sides 123band 124bof cartridge 122 extend to living hinge 125b and align with living hinge channel 169b. In this manner, sutures 129a, 129bmay be guided from lower portion 124 to upper portion 123 on each side of the instrument to form a loop upon clamping of tissue and actuation of the surgical instrument.

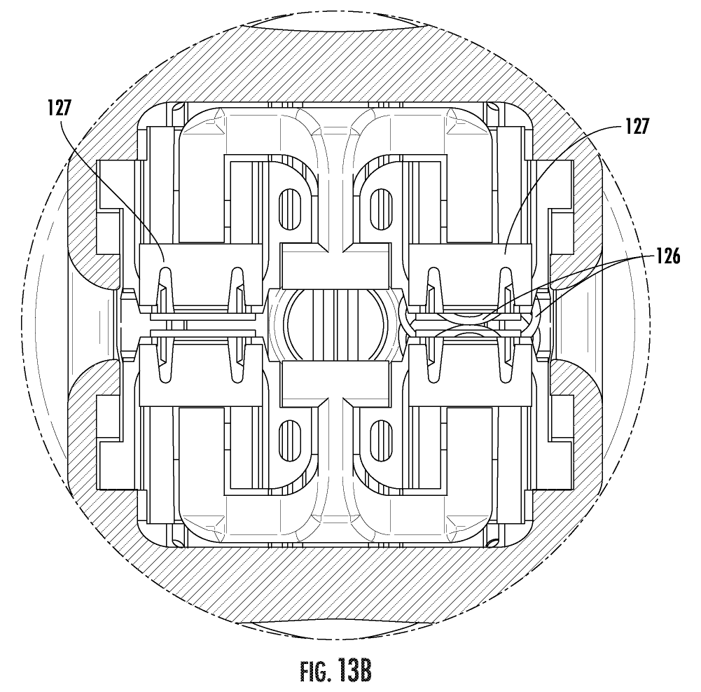

Cartridge 122 also may include a shuttle 130 having an inclined distal portion 131 that, upon distal movement, sequentially acts on staple drivers 127, camming them towards grasped tissue thereby forcing the staples towards grasped tissue. Legs 157 of staples 126 are positioned on either side of sutures 129a, 129b, and are configured to secure sutures 129a, 129b to grasped tissue upon actuation of the surgical instrument. Details of the mechanism for formation of staples 126 to provide purse strings are described below. (See FIGS. 13 and 13A.)

In embodiments, upper portion 123 of cartridge 122 includes distal end portion 190 that, upon closing of jaws 111, 112, overlaps a distal end portion 195 of lower portion 124. Distal end portion 190 prevents cartridge 122 from sliding out of the jaws after installation. Distal end portion 190 may include a flexing latch member 192, that upon installation of cartridge 122 in jaws 111, 112 would engage with a recess (not shown) contained within jaw 111, retaining latch member 192 in place until defeated by application of finger pressure to release it. Flexure would provide an over-center snap feeling between the latched and the unlatched position, such that a surgeon would be able to feel the difference between the latched and unlatched positions. It is envisioned that latch 192 may be held in place by any number of desired mating features, such as a protrusion, or a cutout formed on jaw 111. In an alternative embodiment shown in FIGS. 2C-2D cartridge 122 may further include cutouts 199aconfigured to engage at least one hook 199b contained within jaws 111, 112. Upon installation, cartridge 122 may slightly pivot such that hooks 199bcatch within recesses 199a helping to retain the cartridge 122 in place.

Distal end portions 129c of suture 129a extend beyond the distal ends of suture retaining slots 159a and 159c and distal end portions 129d of suture 129b extend beyond the distal ends of suture retaining slots 159b and 159d. Thus, suture 129amay, for example, extend from distal end portion 190, extending proximally in channel 159a within side 123a of upper portion 123 of cartridge 122, around living hinge 125a through living hinge channel 169a, and then extend distally along suture channel 159c of side 124a of lower portion 124 of cartridge 122. Cartridge 122 is installed in a direction substantially parallel to the longitudinal axis of end effector 110, and is inserted in the direction of arrow A from the distal end of surgical instrument 100 towards the proximal end of surgical instrument 100, as shown in FIG. 2A. In alternative embodiments, it is envisioned that the stapler may allow for installation of cartridge 122 in a radial direction that is substantially orthogonal to the longitudinal axis of end effector 110. FIG. 2E shows an illustrative jaw 112 configured to allow for radial installation of cartridge 122. Hooks 199bon the cartridge may engage recesses 199aformed on jaw 112 to help retain cartridge 122 within jaw 111 in a similar manner as previously described embodiments.

As seen in FIG. 3, when a fresh unfired cartridge 122 is installed, shuttle 130 prevents unwanted separation of sides 123a and 123b of upper portion 123 of cartridge 122 and of sides 124a and 124b of lower portion 124 of cartridge 122. Additionally, cartridge 122 includes protrusions 121 configured to slide into slots 113 formed on jaws 111, 112. Slots 113 of jaws 111, 112 serve as guide rails for protrusions 121 to ensure proper alignment upon installation of a fresh cartridge 122. Additionally, the interaction of cartridge protrusions 121 and slots 113 limits upward and downward movement of cartridge 122 during installation and firing.

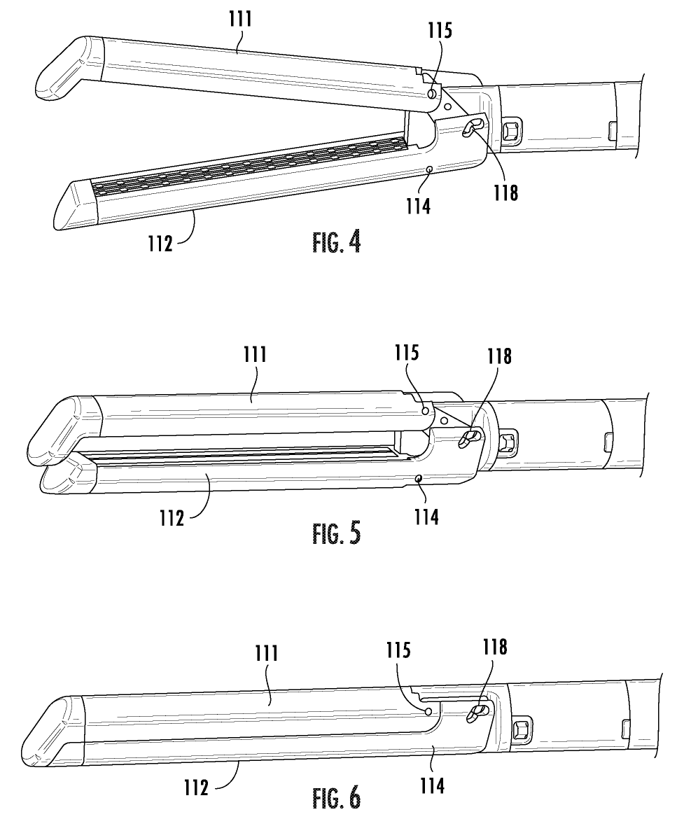

FIGS. 4-6 depict the movement of jaws 111, 112 as the instrument is closed in preparation for actuation.

In FIG. 4, jaws 111, 112 are in an open position, resembling a grasper, and are in an angled-closure zone. Jaws 111, 112 close angularly until they are substantially parallel, as shown in FIG. 5. Once jaws 111, 112 are substantially parallel they continue to close while remaining substantially parallel to compress tissue. Once closed about tissue, the instrument may be fired to cut and insert staples into the grasped tissue, while causing sutures 129a, 129b to be captured by the staples form a purse string around the clamped tissue. The mechanism by which the jaws are opened and closed are described in more detail below.

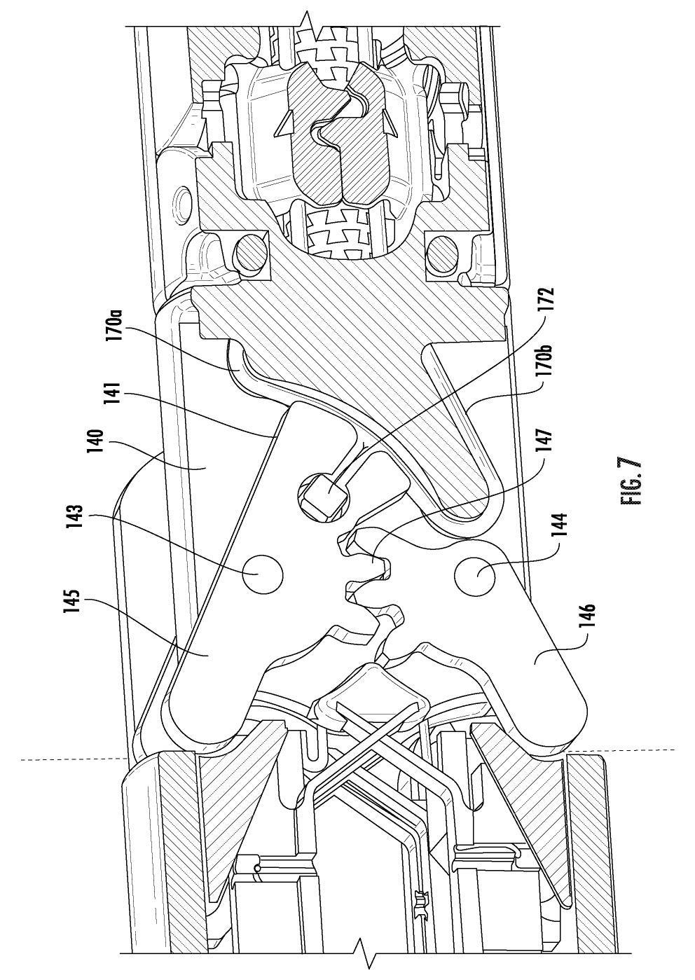

FIG. 7 shows a portion of surgical instrument 100 configured to open and close the jaws including clevis 140, first link member 145, second link member 146, and drive cables 170a, 170b.

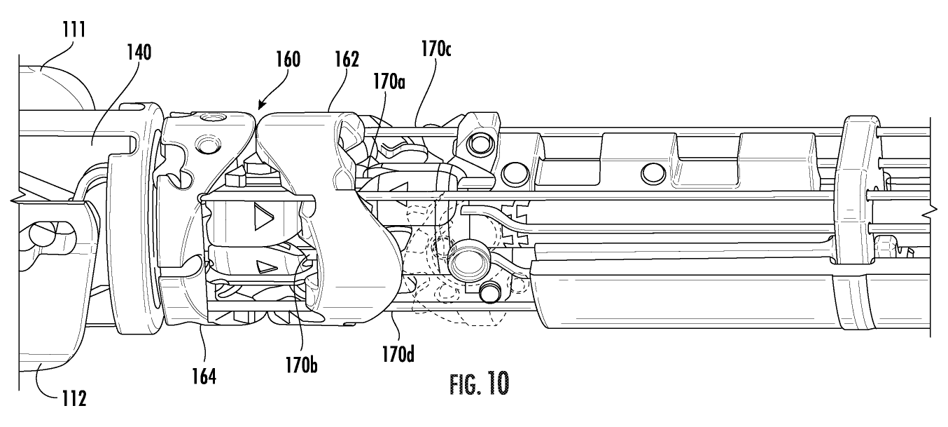

As shown in FIG. 7, two drive cables 170a, 170b route symmetrically to the backend of surgical instrument 100, and terminate in a knot 172 within the proximal end 141 of a first link member 145. As cables 170a, 170b are pulled by the force of the motor (not shown) or via some other mechanism, first link member 145 rotates about pivot pin 143 on clevis 140, causing a second link member 146 to rotate about pivot pin 144 on clevis 140, thereby moving the jaws towards an open or closed position depending on which drive cable was pulled. The resulting jaw force is substantially similar to the cable pull force. One of ordinary skill will appreciate that a pair of drive cables may be present on each side, of surgical instrument 100, each pair of drive cables including a first cable for closing a first link, and a second cable for opening the first link. Using four cables in total for opening and closing of the jaw reduces the stress on each cable, and allows for symmetrically balanced cable forces through the wrist 160 such that actuating the jaw does not impart pitch or yaw forces into the wrist. (See FIG. 10.)

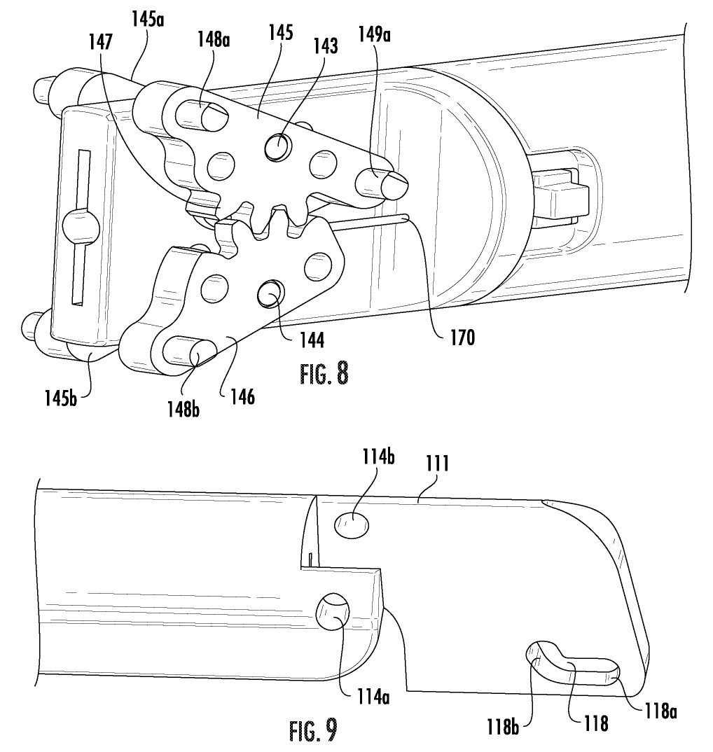

As illustrated in FIGS. 8 and 9, link member 145 includes upper outer pin 148a, and link member 146 includes lower outer pin 148b. Upper outer pin 148ais configured to be positioned within opening 114 of jaw 111, while lower outer pin 148b is configured to be positioned within an opening 115 of jaw 112. (See, e.g., FIGS. 1A and 4-6.) Link member 145 also include cam slot pin149a configured to engage and ride within cam slot 118 formed on jaw 111. Cam slot 118 is designed such that the proximal portion 118a of cam slot 118 allows for parallel closure of jaw 111, while the distal portion 118b of cam slot 118 allows for angular closure of jaws 111 upon rotation of link members 145, 146 due to the force of drive cables 170a, 170b upon actuation of the surgical instrument. A second set of link members 145a, 145band corresponding openings and cam slot, similar to link members 145, 146 is present on the far side of clevis 140 jaws 111, 112 and function similarly to link members 145, 146. Links members 145, 146 also include gear teeth 147 to enforce equally symmetric motion of jaws 111,112.

FIG. 10 illustrates the articulation mechanism including an articulating wrist 160, and drive cables 170c, 170d running from the proximal end of the instrument to wrist links 162, 164. Wrist 160 includes a number of links that provide a desired amount of motion, such as +/- 90 degrees in a pitch or yaw direction. In embodiments, a single joint can provide up to a 90 degree angular deflection. According to an exemplary embodiment, a wrist may include a plurality of links to achieve higher ranges of motion, such as, for example, wrists having a range of motion of up to +/- 180 degrees in a pitch or yaw direction. Typically, actuation elements such as, for example, pull/pull tendons or push/pull rods, and electrical conductors that are connected to a wrist and/or end effector of an instrument may extend through the elongated shaft of the instrument. Further, the actuation elements may extend through the elongated shaft and connect to a transmission mechanism that typically provides a mechanical coupling of the drive tendons to drive motors. As noted above, surgical instrument 100 may include two pairs of drive cables for opening and closing of the jaws, reducing the stress on each cable, and allowing for symmetrically balanced cable forces through wrist 160 such that actuating the jaw does not impart pitch or yaw forces into the wrist. Additionally, it is envisioned that the cables may merge within the mid-section of surgical instrument 100 such that only two cables are presented at the input capstans (not shown). This type of merging allows for a pulley to maintain cable length conservation as the wrist pitches or yaws. Additional details of other joints and wrist actuation elements usable with the embodiments disclosed herein, are disclosed in Int'l. Pub. No. WO 2015/127250A1, the entire disclosure of which is incorporated by reference herein.

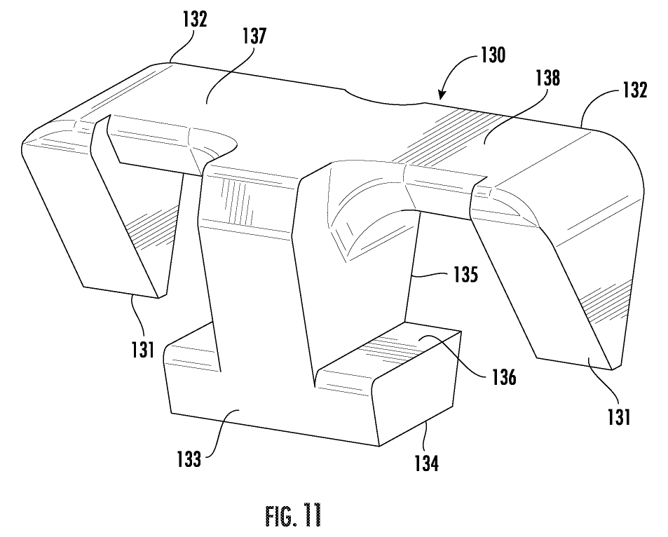

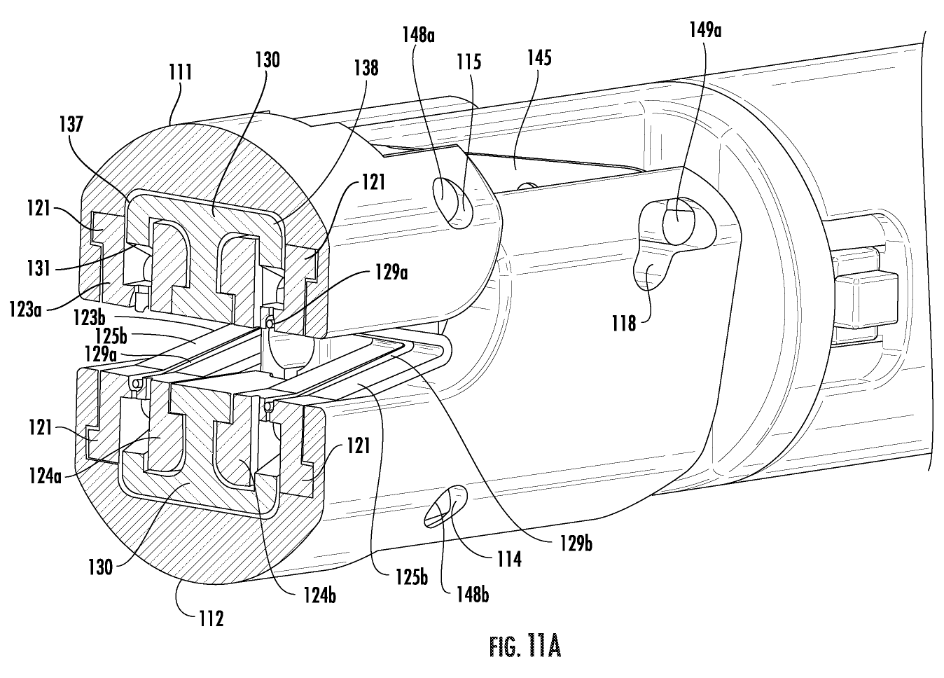

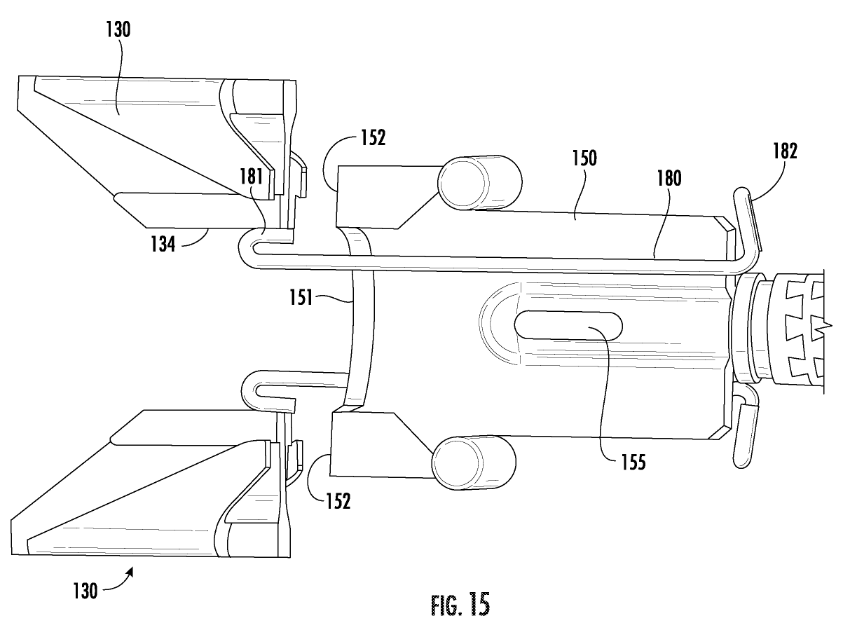

FIGS. 11 and 11A show shuttles 130 contained in cartridge 122. Each shuttle 130 has a body 135 including inclined distal portions 131 and wings 137, 138. A shuttle 130 is positioned in each of upper and lower portions 123, 124 of cartridge 122 and prevent first side 123a and second side 123b of upper portion 123 and first side 124a and second side 124b of lower portion 124 from splaying during handling to ensure that cartridge 122 fits within slots 113 of jaws 111, 112. (See FIG. 11A.) Upon distal movement of shuttle 130, inclined distal portions 131 sequentially act on staple drivers 127, camming them upwardly thereby forcing staples 126 through staple receiving pockets 128. Shuttle 130 also includes proximal faces 132 that may be substantially perpendicular to the axis of end effector 110. Leading edge 133 of shuttle 130 herds tissue toward the center knife channel 153 so that the tissue may be severed by knife 151. (See FIG. 13.) Shuttle 130 also includes inner edge 134 which is described in further detail below. (See FIG. 15.)

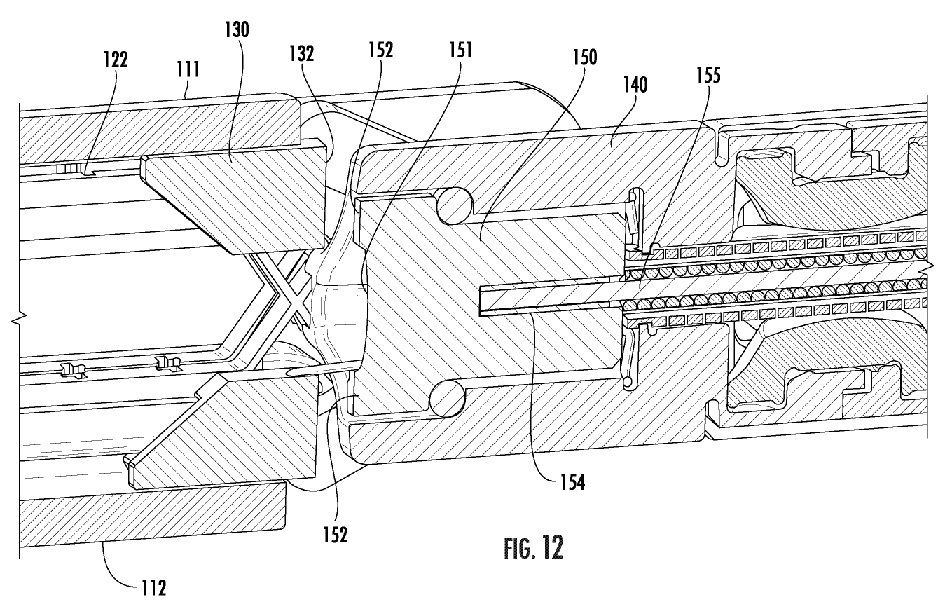

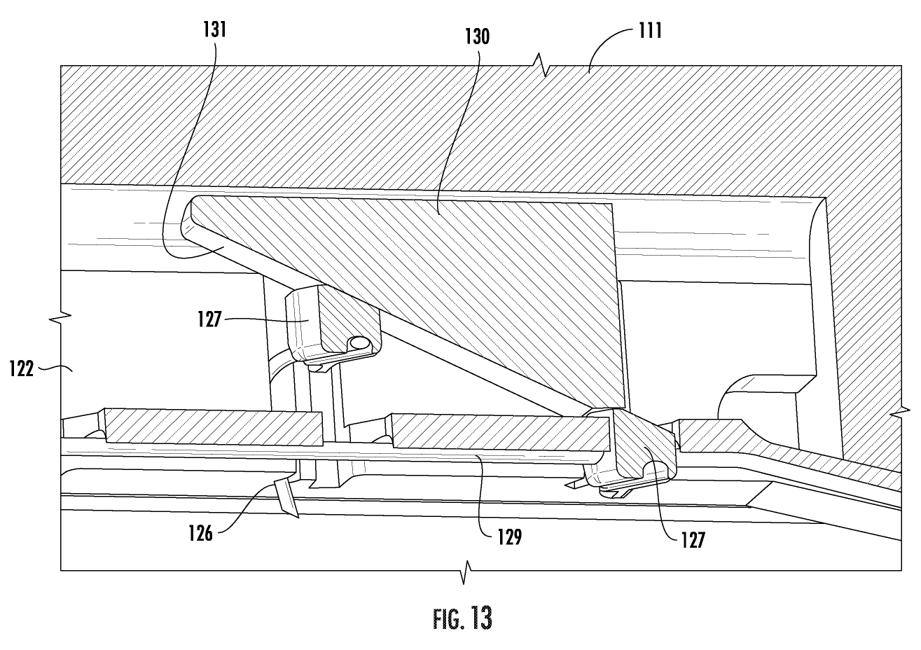

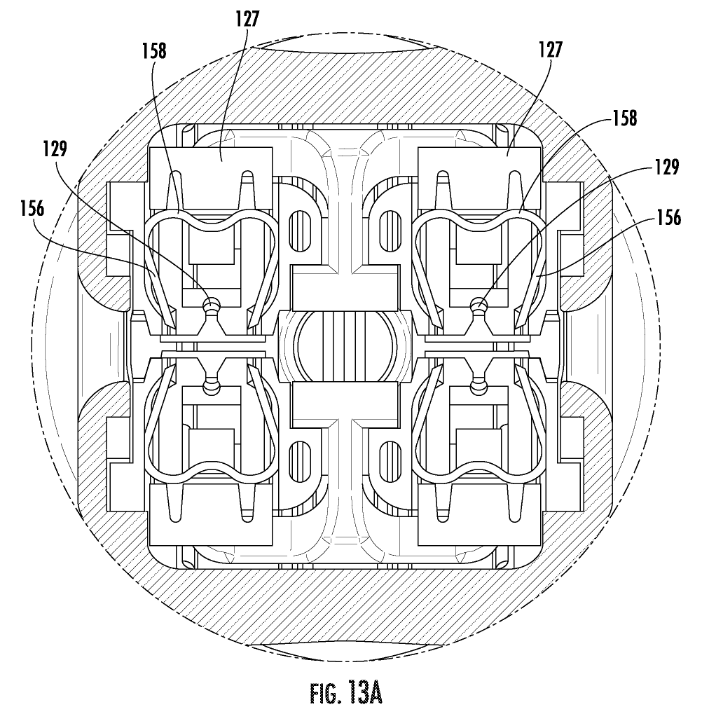

FIGS. 12-13 illustrate distal translation of the components of surgical instrument 100 upon actuation to sever and insert staples into clamped tissue such that a purse string is formed by sutures 129a, 129b.

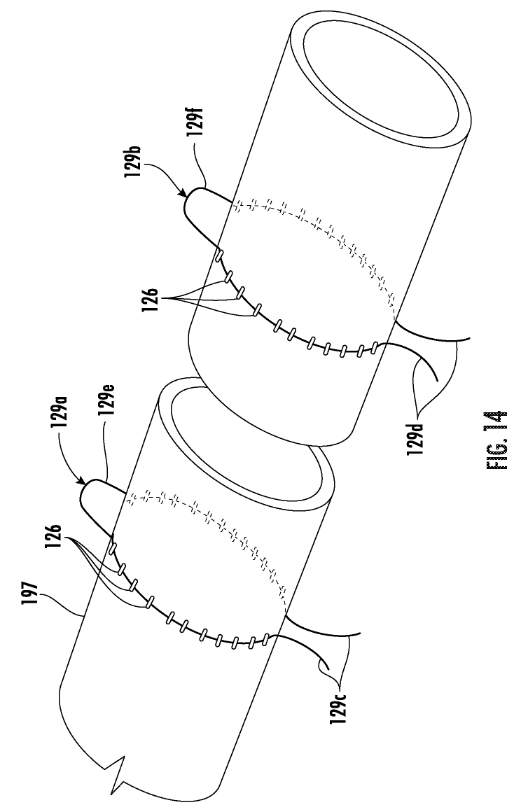

FIGS. 12 shows a drive member 150 having an integrated knife edge 151, shuttles 130, clevis 140, and drive rod 155. Upon actuation of surgical instrument 100, drive member 150 is pushed distally or pulled proximally through center knife channel 153 of cartridge 122 by drive rod 155 that is secured to a proximal cutout 154 formed on drive member 150. Drive member 150 may be coupled to any known actuation mechanisms including manually-activated actuators, motor-driven or powered actuators, or other types of actuation mechanisms. Drive member 150 includes distal edges 152 that are substantially aligned with proximal faces 132 of shuttle 130. Upon actuation of surgical instrument 100, distal edges 152 of drive member 150 engage proximal faces 132 of shuttle 130 translating shuttle 130 distally. Drive member 150 also includes knife edge 151 positioned between distal edges 152 for severing clamped tissue upon distal translation of drive member 150 through center knife channel 153 during firing of surgical instrument 100. The central position of knife channel 153 ensures that knife edge 151 severs tissue that is adjacent to and between the two portions of tissue that are stapled and sutured. (See FIG. 14.)

FIG. 13 shows shuttle 130 traveling distally during firing of surgical instrument 100. In FIG. 13, inclined distal portion 131 of shuttle 130 has fully deployed a first staple 126a and is engaging a second staple driver 127 to deploy a second staple 126b. As seen in FIGS. 13A and 13B, initially, each staple 126 is pushed under a force applied by a staple driver 127 to the backspan 158 of the staple while lateral forces are simultaneously applied against the legs 156 by the lips 157 of staple receiving pockets 128 so that the legs 156 begin to deform and move towards each other while penetrating into tissue 197. As depicted in FIG. 13B near the end of each stroke of staple drivers 127 the legs 156 of a staple have been substantially deformed so as to be in crossing relation to each other. In this position, the staples will not readily pull out from tissue 197. At the same time, sutures 129a,129bare pushed out of channels 159a-d and are captured between the deformed staples 126 and tissue 197. Sutures 129 form a purse string as they are retained within the deformed staples 126.

FIG. 14 depicts an illustrative blood vessel or tissue that has been severed and stapled with a surgical instrument in accordance with this disclosure. Sutures 129a, 129bhave been secured to the tissue in a purse string formation with staples 126 holding sutures 129a, 129b in place. Once secured, another instrument may be used to cinch the purse string tight by pulling on the ends 129c, 129d. In embodiments, a clips 165a, 165b may be preloaded into distal end of cartridge 122 and secured to the ends 129c, 129d of sutures 129a, 129b and used to allow a surgeon to cinch the purse string tight by hand to retain the sutures 129a, 129b in the cinched configuration.

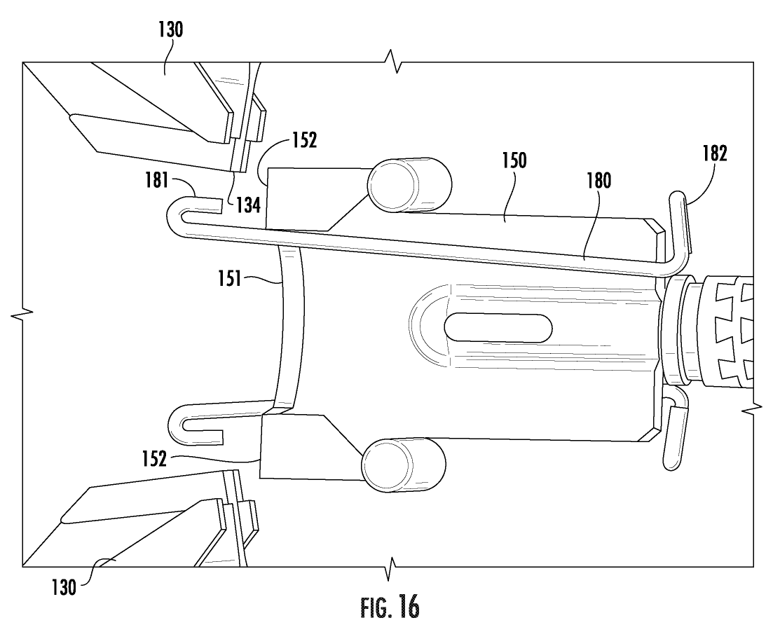

FIGS. 15 and 16 show a lockout mechanism that may be used with surgical instruments in accordance with embodiments of this disclosure.

In FIG. 15, a disabled locking mechanism is shown. Locking member 180 has an engagement portion 181 on the distal end of locking member 180, and a proximal portion 182 on the proximal end of locking member 180. In embodiments, distal portion 181 of locking member 180 is shaped like a hook. In embodiments, locking member 180 is formed from spring wire. When an unfired cartridge is installed, inner edge 134 of shuttle 130 contacts engagement portion 181 of locking member 180, keeping it out of alignment with distal edges 152 of drive member 150. In this position, drive member 150 may be translated distally allowing a user to actuate the instrument.

FIG. 16 shows an enabled locking mechanism. Locking member 180 is designed such that engagement portion 181 is biased towards a position that aligns engagement portion 181 with distal edges 152 of drive member 150. As seen in FIG. 16, when shuttle 130 is moved and inner edge 134 of shuttle 130 no longer contacts locking member 180, engagement portion 181 of locking member 180 moves back to its naturally biased position, where it is aligned with distal edges 152 of drive member 150. Once the instrument is fired and retraction of drive member 150 occurs, a user may not fire the instrument again without installing a fresh unfired cartridge, as distal edges 152 will contact engagement portion 181 of locking member 180 and the drive member 150 will be prevented from translating distally unless a fresh cartridge is installed, and the jaws have closed sufficiently such that they are parallel and clamped on tissue.

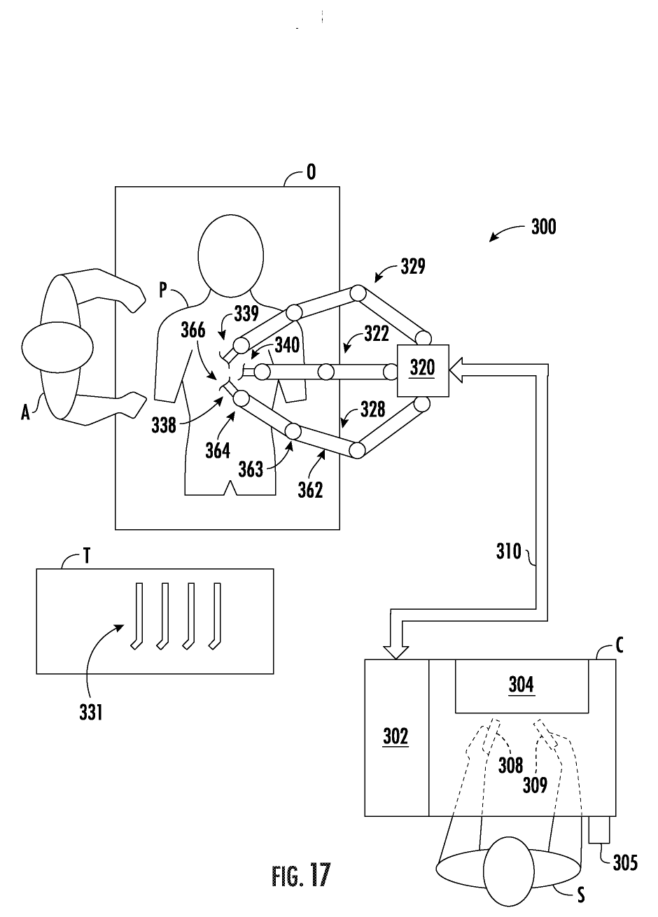

The present surgical instrument for applying one or more purse string sutures may be used in a robotic surgical system. FIG. 17 illustrates, as an example, a top view of an operating room employing a robotic surgical system. The robotic surgical system in this case is a robotic surgical system 300 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The Console includes a monitor 304 for displaying an image of a surgical site to the Surgeon, left and right manipulatable control devices 308 and 309, a foot pedal 305, and a processor 302. The control devices 308 and 309 may include any one or more of a variety of input devices such as joysticks, gloves, trigger-guns, hand-operated controllers, or the like. The processor 302 may be a dedicated computer that may be integrated into the Console or positioned next to it.

The Surgeon performs a minimally invasive surgical procedure by manipulating the control devices 308 and 309 (also referred to herein as “master manipulators”) so that the processor 302 causes their respectively associated robotic arm assemblies, 328 and 329, (also referred to herein as “slave manipulators”) to manipulate their respective removably coupled surgical instruments 338 and 339 (also referred to herein as “tools”) accordingly, while the Surgeon views the surgical site in 3-D on the Console monitor 304 as it is captured by a stereoscopic endoscope 340.

Each of the tools 338 and 339, as well as the endoscope 340, may be inserted through a cannula or other tool guide (not shown) into the Patient so as to extend down to the surgical site through a corresponding minimally invasive incision such as incision 366. Each of the robotic arms is conventionally formed of links, such as link 362, which are coupled together and manipulated through motor controlled or active joints, such as joint 363.

The number of surgical tools used at one time and consequently, the number of robotic arms being used in the system 300 will generally depend on the diagnostic or surgical procedure and the space constraints within the operating room, among other factors. If it is necessary to change one or more of the tools being used during a procedure, the Assistant may remove the tool no longer being used from its robotic arm, and replace it with another tool 331 from a Tray (“T”) in the operating room.

The monitor 304 may be positioned near the Surgeon's hands so that it will display a projected image that is oriented so that the Surgeon feels that he or she is actually looking directly down onto the operating site. To that end, images of the tools 338 and 339 may appear to be located substantially where the Surgeon's hands are located.

The processor 302 performs various functions in the system 300. One important function that it performs is to translate and transfer the mechanical motion of control devices 308 and 309 to their respective robotic arms 328 and 329 through control signals over bus 310 so that the Surgeon can effectively manipulate their respective tools 338 and 339. Another important function is to implement various control system processes as described herein.

Although described as a processor, it is to be appreciated that the processor 302 may be implemented in practice by any combination of hardware, software and firmware. Also, its functions as described herein may be performed by one unit, or divided up among different components, each of which may be implemented in turn by any combination of hardware, software and firmware.

For additional details on robotic surgical systems, see, e.g., U.S. Pat. Nos. 6,493,608 and 6,671,581, the entire contents of which are incorporated herein by this reference.

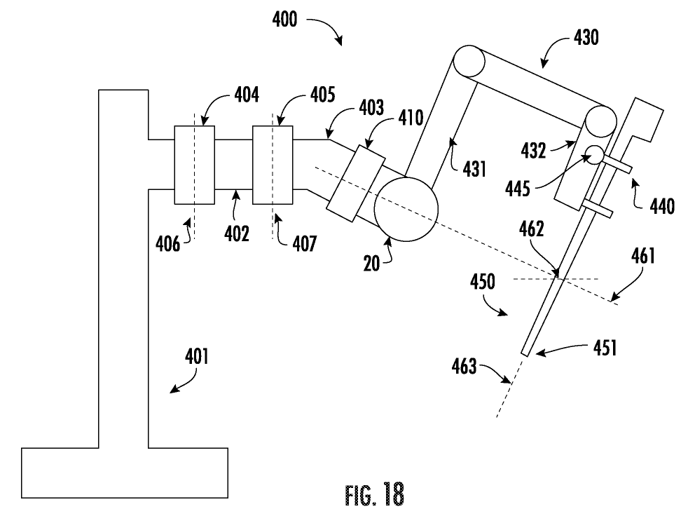

FIG. 18 illustrates, as an example, a side view of a simplified (not necessarily in proportion or complete) illustrative robotic arm assembly 400 (which is representative of robotic arm assemblies 328 and 329) holding a surgical instrument 450 (which is representative of tools 338 and 339) for performing a surgical procedure. The surgical instrument 450 is removably held in tool holder 440. The arm assembly 400 is mechanically supported by a base 401, which may be part of a patient-side movable cart or affixed to the operating table or ceiling. It includes links 402 and 403 which are coupled together and to the base 401 through setup joints 404 and 405.

The setup joints 404 and 405 in this example are passive joints that allow manual positioning of the arm 400 when their brakes are released. For example, setup joint 404 allows link 402 to be manually rotated about axis 406, and setup joint 405 allows link 403 to be manually rotated about axis 407.

Although only two links and two setup joints are shown in this example, more or less of each may be used as appropriate in this and other robotic arm assemblies in conjunction with the present invention. For example, although setup joints 404 and 405 are useful for horizontal positioning of the arm 400, additional setup joints may be included and useful for limited vertical and angular positioning of the arm 400. For major vertical positioning of the arm 400, however, the arm 400 may also be slidably moved along the vertical axis of the base 401 and locked in position.

The robotic arm assembly 400 also includes three active joints driven by motors. A yaw joint 410 allows arm section 430 to rotate around an axis 461, and a pitch joint 420 allows arm section 430 to rotate about an axis perpendicular to that of axis 461and orthogonal to the plane of the drawing. The arm section 430 is configured so that sections 431 and 432 are always parallel to each other as the pitch joint 420 is rotated by its motor. As a consequence, the instrument 450 may be controllably moved by driving the yaw and pitch motors so as to pivot about the pivot point 462, which is generally located through manual positioning of the setup joints 404 and 405 so as to be at the point of incision into the patient. In addition, an insertion gear 445 may be coupled to a linear drive mechanism (not shown) to extend or retract the instrument 450 along its axis 463.

Although each of the yaw, pitch and insertion joints or gears, 410, 420 and 445, is controlled by an individual joint or gear controller, the three controllers are controlled by a common master/slave control system so that the robotic arm assembly 400 (also referred to herein as a “slave manipulator”) may be controlled through user (e.g., surgeon) manipulation of its associated master manipulator.

While several embodiments have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus, the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

1. A surgical stapler for applying a suture to tissue, the surgical stapler comprising:
a shaft having distal and proximal ends;
an end effector having a first jaw and a second jaw;
a cartridge removably coupled to the end effector and having a channel for
receiving a suture and a shuttle configured to translate longitudinally through the channel;
a drive member configured to translate distally through the channel of the cartridge to apply staples and the suture to tissue; and
a lockout member movable between a disabled position permitting distal translation of the drive member and an enabled position preventing distal translation of the drive member.

2. The surgical stapler of claim 1, wherein the first jaw comprises a cavity for receiving
the cartridge, and the lockout member comprises an elongate member extending longitudinally through at least a portion of the cavity.

3. The surgical stapler of claim 2, wherein the elongate member comprises a distal end
portion, wherein the shuttle is configured to contact and engage the distal end portion of the lockout member to maintain the lockout member in the disabled position.

4. The surgical stapler of claim 3, wherein the distal end portion of the elongate member
comprises a curved portion and a proximally extending portion configured to contact and engage the shuttle in the disabled position.

5. The surgical stapler of claim 3, wherein the distal end portion of the elongate member
is biased laterally outward from a longitudinal axis of the end effector.

6. The surgical stapler of claim 5, wherein the shuttle disengages from the lockout
member when the shuttle is translated distally through the end effector to allow the lockout member to move into the enabled position.

7. A surgical stapler for applying a suture to tissue, the surgical stapler comprising:
a shaft having distal and proximal ends;
an end effector having a first jaw and a second jaw;
a cartridge removably coupled to the end effector and comprising a first portion having a first channel for receiving a suture and a second portion having a second channel for receiving a suture;
a first shuttle configured to translate longitudinally through the first channel;
a second shuttle configured to translate longitudinally through the second channel; and
a drive member configured to translate distally through the first and second
channels of the cartridge to apply staples and a suture to tissue.

8. The surgical stapler of claim 7, wherein the first jaw comprises a cavity for receiving
the first portion of the cartridge and the second jaw comprises a cavity for receiving the second portion of the cartridge.

9. The surgical stapler of claim 8, wherein the first portion of the cartridge comprises a
first staple and a first staple driver, wherein the first shuttle comprises an inclined portion that, upon distal movement of the first shuttle, cams the first staple driver towards the tissue.

10. The surgical stapler of claim 9, wherein the second portion of the cartridge comprises
a second staple and a second staple driver, wherein the second shuttle comprises an inclined portion that, upon distal movement of the second shuttle, cams the second staple driver towards the tissue.

11. The surgical stapler of claim 10, wherein the suture extends through the first and
second staples such that the suture, in combination with the first and second staples, forms a purse string with the tissue.

12. The surgical stapler of claim 8, wherein the first and second portions of the cartridge
each comprise a central channel, a first lateral channel disposed laterally outward from a first side of the central channel and a second lateral channel disposed laterally outward from a second opposing side of the central channel, wherein the first and second shuttles are configured to translate through the central channels of the first and second portions of the cartridge.

13. A surgical stapler for applying a suture to tissue, the surgical stapler comprising:
A shaft having distal and proximal ends;
an end effector having a first jaw and a second jaw;
a cartridge removably coupled to the end effector and comprising a first portion having a first channel for receiving a suture and a second portion having a second channel for receiving a suture;
a first shuttle configured to translate longitudinally through the first channel;
a second shuttle configured to translate longitudinally through the second channel;
a drive member configured to translate distally through the first and second
channels of the cartridge to apply staples and a suture to tissue; and
a lockout member movable between a disabled position permitting distal translation of the drive member and an enabled position preventing distal translation of the drive member.

14. The surgical stapler of claim 13, wherein the first and second shuttles engage the
lockout member to hold the lockout member in the disabled position.

15. The surgical stapler of claim 14, wherein the first and second shuttles disengage from
the lockout member when the first and second shuttles are translated distally through the end effector to allow the lockout member to move into the enabled position.

16. The surgical stapler of claim 13, wherein the first jaw comprises a cavity for receiving
the first portion of the cartridge and the second jaw comprises a cavity for receiving the second portion of the cartridge.

17. The surgical stapler of claim 16, wherein the lockout member comprises a first elongate
member extending longitudinally through at least a portion of the cavity of the first jaw and a second elongate member extending longitudinally through at least a portion of the cavity of the second jaw.

18. The surgical stapler of claim 17, wherein the first and second elongate members each
comprise a distal end portion, wherein the first shuttle is configured to contact and engage the distal end portion of the first elongate member and the second shuttle is configured to contact and engage the distal end portion of the second elongate member to maintain the lockout member in the disabled position.

19. The surgical stapler of claim 18, wherein the distal end portion of the first and second
elongate members each comprise a curved portion and a proximally extending portion configured to contact and engage the first and second shuttles in the disabled position.

20. The surgical stapler of claim 19, wherein the distal end portion of each of the first and
second elongate members is biased laterally outward from a longitudinal axis of the end effector.
